(19) United States
(12) Patent Application Publication
(10) Pub. No.: US 2008/0103111 A1
(43) Pub. Date: May 1, 2008

(54) Smoking Cessation Treatment with Appetite Suppression

(76) Inventor: Harlan Clayton Bieley, West Palm Beach, FL (US)

Correspondence Address: JOHN C. SMITH, ESQ., 2499 GLADES ROAD, SUITE 113, BOCA RATON, FL, 33431, US

(21) Appl. No.: 11/554,364

(22) Filed: Oct. 30, 2006

Related U.S. Application Data
(60) Provisional application No. 60/767,546, filed on Jun. 21, 2006.

(51) Int. Cl. A61K 31/714; A61K 31/525; A61K 31/51; A61K 31/385; A61K 31/375; A61K 31/198; A61K 31/405

(52) U.S. Cl. 514/52; 514/350; 514/342; 514/440; 514/276; 514/567; 514/554; 514/419; 514/562; 514/561; 514/474; 514/251; 514/250

(57) ABSTRACT

A multi-component compound for the simultaneous treatment of nicotine addiction and the side effects of nicotine withdrawal, such as excessive appetite. The first component is a bivalent negative sulfur compound in an amount effective to control nicotine craving or the withdrawal symptoms resulting from nicotine withdrawal. The bivalent negative sulfur is selected from a group that includes, but is not limited to, alkyl sulfides, colloidal sulfur, hydropersulfides, organic thio compounds or their salts. The second component is a serotonin precursor, such as tryptophan or its derivative 5-HTP, which is used to assist the body in producing more serotonin which in turn suppresses appetite. The appetite suppressant(s) are combined with the bivalent negative sulfur compound(s) to provide a single compound that reduces nicotine craving and simultaneously suppresses increased appetite resulting from nicotine withdrawal.

### BACKGROUND OF THE INVENTION

1. Technical Field

The present invention relates to treatments for individuals seeking to cease smoking. In particular, it relates to a method and treatment for cessation of smoking while simultaneously avoiding unnecessary weight gain.

2. Background

The health problems associated with tobacco smoking have caused numerous individuals to attempt to end their use of tobacco products. However, as is well-known, tobacco products can be highly addictive due to the addictive quality of nicotine which is present in tobacco products. Many individuals attempt to quit smoking by simply stopping their use of tobacco products. Unfortunately, due to the strong addictive nature of nicotine, the abrupt cessation of tobacco usage (i.e., quitting “cold turkey”) fails in most cases. Quite often, individuals require more than willpower alone to free themselves from nicotine addiction. It would be desirable to have a method of aiding an individual to cease smoking without having to rely on substantial levels of willpower.

In addition to the difficulty related to cessation of smoking, attempts to stop smoking often result in the creation of secondary problems related to smoking. In particular, a fairly common side effect that individuals experience when attempting to quit smoking is a substantial increase in appetite. The increase in appetite results in undesirable increases in body weight. In turn, the extra body weight can result in a variety of unwanted health problems, as well as an undesirable change in the individual's appearance. Further, the undesirable increase in body weight can have a detrimental effect on an individual's willpower when attempting to quit smoking, because the additional weight can be very frustrating to an individual. In fact, it may even contribute to an individual's decision to abandon their attempt to quit smoking. It would be desirable to have a method of increasing an individual's chances of success by avoiding unwanted weight gain during the difficult process of overcoming addiction to nicotine.

To assist individuals attempting to quit smoking and free themselves of addiction to nicotine, a variety of methods have been tried. One such method has been the use of nicotine supplements to reduce nicotine craving by the individual. Nicotine supplements can take several forms. For example, a nicotine chewing gum has been developed which permits individuals to satisfy the body's craving for nicotine without damaging their lungs by inhaling tobacco smoke. Likewise, nicotine patches which adhere to an individual's skin for transdermal absorption of nicotine have also been developed. The object of nicotine supplements is to satisfy an individual's craving for nicotine, without the highly negative health consequences of inhaling tobacco smoke. While nicotine supplements protect the individual from the significant harm that smoking causes by satisfying the need to obtain nicotine without inhaling tobacco smoke, they do not overcome the individual's addiction to nicotine. In addition, once the chewing gum is discarded, or the transdermal patch is no longer used, the craving for tobacco products often returns. As a result, nicotine supplements solve only some of problems associated with smoking. It would be desirable to have a method of weaning an individual away from smoking tobacco, while at the same time minimizing the numerous undesirable side effects of a withdrawal from smoking.

More recently, alternative treatments which do not use nicotine supplements have been developed to assist individuals in breaking the tobacco habit. Nicotine is a neural chemical which attaches to specific cell receptor sites in the brain. Therefore, preventing its ability to attach to these cell receptor sites is one method used to interfere with the addictive qualities of nicotine. One such alternative treatment involves the use of chemical compounds which bind to these cell receptor sites, and thereby prevent nicotine from binding to those cell receptor sites. As a result, the nicotine based cravings of the individual are reduced. A number of anti-smoking compounds have been found to be effective for this purpose. These compounds include alkyl sulfides, colloidal sulfur, hydropersulfides, organic thio compounds or their salts. The preferred thio compounds are thioglycerols, thioglycols or their salts.

One example of a commercially available product containing at least one of these compounds is Sulfonil™. Sulfonil is described in U.S. Pat. No. 4,596,706 as a method of controlling craving for tobacco or controlling tobacco withdrawal symptoms. The product uses bivalent negative sulfur compounds which attach to the individual's cell receptor sites, and thereby prevent nicotine from binding to those same cell receptor sites.

By removing or reducing the ability of the nicotine to bind with cell receptor sites, the smoker can gradually eliminate the addiction to nicotine. However, while products such as Sulfonil help an individual to eliminate the nicotine craving, it does nothing to address the side effects of nicotine withdrawal, namely excessive appetite and unwanted weight gain. It would be desirable to have a product which helps an individual overcome nicotine addiction, and also helps an individual avoid the unwanted side effects of nicotine withdrawal.

While the prior art has addressed some of the problems related to cessation of smoking, it has failed to address other problems, such as excessive weight gain, which can occur during the course of withdrawal from nicotine addiction. It would be desirable to have a product which is capable of addressing the basic problem of withdrawal from nicotine addiction, as well as subsequent withdrawal related side effects such as excessive weight gain which in turn leads to other health problems.

### SUMMARY OF THE INVENTION

The present invention relates to a compound for the simultaneous treatment of nicotine addiction and undesirable side effects such as excessive appetite and weight gain that occur during the nicotine withdrawal process. The first component of the compound uses at least one bivalent negative sulfur, in an amount effective to control the craving or the withdrawal symptoms resulting from nicotine withdrawal. The bivalent negative sulfur is selected from a group that includes, but is not limited to, hydropersulfides, alkyl sulfides, colloidal sulfur, organic thio compounds or their pharmaceutically acceptable salts. The most effective thio compounds have proven to thioglycerols, thioglycols or their pharmaceutically acceptable salts.

The second component of the compound relates to cravings, appetite suppression, and control of blood sugar. In the preferred embodiment, appetite suppression is accomplished using amino acids derived from tryptophan, such as 5-HTP.

5-HTP and/or related tryptophan derivatives. These are known in the art to suppress appetite and cravings for certain carbohydrates when ingested. The appetite suppression compounds are combined with the bivalent negative sulfur compound(s) to provide a single compound that produces the desirable effect of reducing nicotine craving while simultaneously suppressing increased appetite which is a result of nicotine withdrawal.

These compounds are combined with suitable binders to form pharmaceutical capsules that can be administered orally. Alternatively, these compounds can be administered via injection, via transdermal cream, gel or patch, via gum or lozenge, or by any other suitable means. In some administration methods, such as gum or lozenge, sweetening agents, such as sugar alcohols, can be added to sweeten the taste without effecting blood sugar levels. Sugar alcohols, such as xylitol, can be used in combination with gums or lozenges to provide sweetness with a lower carbohydrate level than a comparative amount of sugar. As a result, it will not elevate blood sugar levels as much as sugar will. In addition, stevia and agave may also be used as sweeteners. Stevia and agave can both be useful in controlling blood sugar levels.

A therapeutic dosage level is approximately 120 milligrams, twice a day, of the bivalent negative sulfur, and multiple doses of the bivalent negative sulfur may be taken at the same time. Regarding 5-HTP, typical doses would run from a low dosage level of 50 milligrams to a high dosage level of 900 milligrams per day. It is important to note that the dosage level of each component of the compound can vary based on the size/weight of the individual in question, vary based on the tolerance level of that individual for components of the compound, or vary based on the intensity of the craving for nicotine or food. As a result, a small individual, or an individual sensitive to the compound, may achieve adequate results with small dosage levels of 5-HTP (e.g., less than 50 milligrams) per day. Likewise, a large individual, or an individual with a high tolerance level for the compound, may safely take more than what would typically be considered the maximum dosage of 5-HTP (e.g., greater than 900 milligrams) per day.

The treatment uses a compound which addresses both nicotine craving, and appetite control. The first component of the compound is designed to interfere with the ability of nicotine to attach to cell receptor sites, thereby reducing nicotine craving. The other component of the compound is directed to both nicotine craving and appetite control, thereby reducing excessive weight gain associated with drawal from nicotine addiction. The various components of the compound act together to assist an individual to quit smoking without the undesirable side effects of nicotine withdrawal, such as increased appetite and unwanted weight gain.

An advantage of tryptophan and tryptophan derivatives derives from the fact that when the body processes a tryptophan derivative such as 5-HTP, the tryptophan derivative is used by the body as a serotonin precursor to increase serotonin levels. It is the increased serotonin level that decreases appetite and craving for food which lead to excessive weight gain. An added benefit provided by serotonin is that it tends to decrease many types of craving, including craving for nicotine. As a result of its general affect on craving, it not only helps suppress appetite which leads to unwanted weight gain, but it also works synergistically with the sulfur based compounds to decrease craving for nicotine.

### DESCRIPTION OF THE PREFERRED EMBODIMENT

Prior to discussing detailed aspects of the invention, a general overview will be provided. The invention provides an improvement over prior art sulfur-based nicotine withdrawal products which block nicotine from binding with cell receptor sites. The prior art has focused its attention on the use of a variety of sulfur based compounds to treat nicotine addiction. However, it has not placed significant emphasis on the side effects associated with nicotine withdrawal. In particular, a frequent side effect associated with smoking cessation is a marked increase in appetite. The increase in appetite often results in an individual having the health benefits associated with cessation of smoking offset by the health hazards produced by excessive weight gain. These are increased insulin levels which can lead to insulin resistance, increased blood pressure, and unfavorable changes in lipid profiles.

The present invention overcomes these problems by providing a single therapeutic compound which simultaneously reduces nicotine craving to assist an individual to cease smoking, and simultaneously suppresses the individual's appetite to avoid unnecessary and undesirable side effects, such as weight gain, associated with the cessation of smoking.

The invention takes advantage of known sulfur based compounds which interfere with the binding of nicotine with cell receptor sites. These compounds, and their method of use, are discussed in detail in U.S. Pat. Nos. 4,416,869 and 4,596,706, which are incorporated by reference herein in their entirety. Applicant's invention improves over these prior art inventions by adding compounds which are specifically designed to reduce the increased appetite which is caused by nicotine withdrawal. Further, the compounds selected to reduce appetite also enhance the body's ability to suppress nicotine cravings. As a result, an individual seeking to cease smoking will not have the additional problems generated by the common side effect of nicotine withdrawal, namely, increased appetite and weight gain. Applicant's invention provides a single dose which is directed to both problems: overcoming nicotine addiction and reducing the side effect of increased appetite during nicotine withdrawal.

Having discussed the features and advantages of the invention in general, we turn now to a more detailed discussion of the figures.

As discussed above, the U.S. Pat. Nos. 4,416,869 and 4,596,706 teach a variety of sulfur based compounds which are useful in the treatment of nicotine addiction. In particular, these patents teach the use of bivalent negative sulfur compounds which are useful for reducing nicotine craving in individual's seeking to cease smoking tobacco products. However, these products do not effectively address side effects related to the cessation of smoking, most notably: unwanted weight gain due to increased appetite. The advantage of Applicant's invention is that it allows an individual to cease smoking without this undesirable side effect.

Applicant's invention provides a new compound which incorporates known compounds in the prior art related to suppression of nicotine addiction with a second compound which combined with those compounds to simultaneously suppress the unwanted increase in appetite which occurs during nicotine withdrawal. Applicant's invention uses derivatives of the amino acid tryptophan to reduce appetite so that an individual can cease smoking tobacco products without the added stress and health hazards related to excessive weight gain.

In the preferred embodiment, both the compounds related to nicotine addiction, and the amino acids used for appetite suppression are combined into a single dose for ingestion by the individual. Those skilled in the art will recognize that these compounds can be administered via a variety of methods, such as pills/capsule ingestion, liquid ingestion, intramuscular injection, intravenous injection, nasal spray, or via inhaler. When used as a liquid, the compound can be administered in combination with any suitable solution, liquid carrier, liquid medium, etc. The only requirement is that liquid selected is suitable for use with the components of the compound. However, while a variety of administration methods can be used (e.g., injection, transdermal patches creams, gels, lozenges, gums, etc.), the preferred embodiment envisions the simple and convenient process of administering the compound via a pill or capsule.

In the preferred embodiment, the tryptophan derivative used by the Applicant is 5-hydroxytryptophan (“5-HTP”), which is a nutrient. It is derived from the amino acid L-Tryptophan. L-Tryptophan plays a vital role in our health. Tryptophan an essential amino acid for building proteins and enzymes, and serves as the precursor for serotonin, and the hydrogen carriers NADH and NADPH. 5-HTP functions as a precursor to serotonin (5-HT, 5-hydroxytryptamine). Serotonin, a neurotransmitter, plays an important role in regulation of mood, appetite, body temperature, and the secretion of various hormones. While serotonin does not readily cross the blood brain barrier, serotonin precursors such as L-tryptophan and 5-HTP can. Supplementation with these precursors increases levels of serotonin. In addition, 5-HTP is more efficient than L-tryptophan because it bypasses the rate-limiting step of serotonin synthesis (tryptophan hydroxylase).

Tryptophan has a variety of side effects when ingested by humans. It can be used as a mood-enhancer, and it can help individuals sleep. In fact, it has been widely used to treat insomnia and depression. It can increase pain tolerance, and it can also reduce appetite. An advantage of 5-HTP over some other appetite suppressants is that it has small molecule size. The 5-HTP accesses the brain from the bloodstream, and once in the brain, it can be converted into serotonin. It is the serotonin, created from the 5-HTP, acting on the different serotonin receptor sites, which ultimately acts as an appetite suppressant, and helps to reduce cravings for nicotine. A further advantage of 5-HTP over other potential appetite suppressants is that it is a naturally occurring compound which is produced in the body from tryptophan which is found in high-protein foods such as beef, chicken, fish, and/or dairy products. A normal variety of food products will include many items that are rich in tryptophan.

In the preferred embodiment, the dosage level of 5-HTP ranges from 50 mg per day up to 900 mg per day. 700 to 900 mg of 5-HTP would normally be considered a high dose. Of course, those skilled in the art will recognize that there are many factors that influence the appropriate dosage level for an individual. For example, the size, weight and tolerance of individuals can vary widely. Therefore, an appropriate dosage for one individual may not be safe for another. A large individual may have the ability to use dosage levels well in excess of 900 mg. Some individuals may also have high tolerance levels for particular compounds which will result in the ability to use high dosage levels. Of course the opposite will be true for individuals who are small or particularly sensitive to a given compound. In addition, other factors related to an individual may create higher or lower levels of appetite which would necessitate the change in dosage levels. As a result, unique factors related to each individual should be taken into account when determining the proper levels of each of the components of the compound.

In the preferred embodiment, the compound would be taken twice a day, to maintain stable levels of nicotine suppressant. In addition, it has been found that the appetite suppressant is more effective if taken before or after meals, because it is absorbed by the body more rapidly if taken on an empty stomach. As a result, an individual would preferably take the compound approximately an hour before meals, or approximately 2 hours after meals. Typically, an individual would take a dose twice a day. This would maintain relatively stable levels of nicotine suppressant throughout the day, and will also suppress hunger at the appropriate times.

Because 5-HTP is a precursor used by the body to produce serotonin which suppresses appetite, its use in combination with the aforementioned nicotine suppressant allows an individual to improve the chances of successfully overcoming a tobacco habit. This is because the individual will not have negative side effects such as increased weight gain, which may in fact frustrate the individual to the point where they resume smoking. Due to that, the individual is more likely to succeed when attempting to quit smoking. Of course, avoiding unnecessary weight gain provides many advantages for the overall health of the individual. In addition, because serotonin also inhibits cravings for nicotine, it will synergistically enhance the sulfur based compounds which are directed specifically at reducing nicotine cravings.

In addition to the use of 5-HTP to suppress appetite, other additives may be included in the compound to enhance weight control. For example, there are a number of nutritional supplements which enhance the body's ability to metabolize fat, such as vitamin B12, inositol, methionine, and choline. Additives such as this complement the suppressant of appetite by enhancing the body's ability to metabolize fat. Therefore, these additives would also help reduce the risk of weight gain during smoking cessation.

Inositol, in all of its forms, may be helpful in more than one way. Higher doses may help increase GABA and reduce anxiety. The prior art suggests that Inositol may increase neurotransmitters such as serotonin, and GABA. While medical literature suggests that individuals may safely ingest 1-18 grams per day, the preferred embodiment envisions a dosage level of approximately 1 gram/day.

Another effective additive is Dopamine, which is a neurotransmitter. L-Tyrosine 100-200 mg twice a day as well as N-acetyl tyrosine 100 mg twice a day and L or DL-phenylalanine 300 mg a day will increase dopamine along with the B-Vitamins. The addition of the neurotransmitter precursor of dopamine may also help decrease cravings for certain carbohydrates like sweets, but also salt and fat. The L-phenylalanine form may work best.

Other optional additives can also be usefully combined with the compound. For example, alpha-lipoic acid helps reduce cravings for sweets, which can contribute significantly to weight gain. Likewise, chromium picolinate, the chromium product, chromium polynicotinate™, and biotin can be helpful in regulating blood sugar metabolism and may be included in the compound. Research suggests that chromium may help prevent glucose-induced elevation of systolic blood pressure and decreased measures of lipid peroxidation. High serum glucose levels which can also contribute to a destructive process known as glycosylation, also called nonenzymatic glycation, in which glucose molecules bind to proteins and interfere with their function. People with diabetes have a highly increased rate of protein glycosylation and this plays a major part in their increased risk of atherosclerosis and many other diseases. For many with diabetes, chromium enhances the ability of insulin to lower serum glucose levels. In addition to these nutritional supplements, some vitamins, mainly B6, may be used by the body in the process of converting 5-HTP to serotonin. Therefore, their inclusion in the compound may increase the efficiency of conversion which will result in greater serotonin levels. Vitamin B6 also acts as a diuretic which further helps to control weight.

In the preferred embodiment, two size 00 capsules are taken, twice a day, with each capsule containing the following doses:

1.100 mg 5HTP
2. 60 mg Thioglycerol
3. 25 mg Choline Citrate
4. 25 mg Pantothenic Acid (as D-Calcium Pantothenate)
5. 200 mg Inositol
6.25 mg L-methionine
7. 50 mg Alpha Lipoic Acid
8. 50 mg Vitamin B-6 (as Pyridoxine HCl)
9. 50 mg Vitamin C
10. 200 mcg Folic Acid
11. 50 mcg Vitamin B12 (as Methylcobalamin)
12. 25 mg Magnesium Citrate

Optionally, the following may be added to the foregoing:

13. 200-400 mg L-Tyrosine
14. 200 mg N-acetyl tyrosine
15. 300 mg L or DL-phenylalanine

Those skilled in the art will recognize that while the aforesaid formula is preferred, changes can be made to the formula without losing its overall effectiveness. Two components of the compound are the most important. The first component is 5-HTP which is used to suppress appetite. In the preferred embodiment, this is administered in an approximate dosage of 200 mg. The second compound is Thioglycerol, which is a sulfur based compound that is used to block nicotine receptor cells. By blocking the receptor cells, nicotine cravings are reduced. The Thioglycerol is typically administered in an approximate dosage of 120 mg. The combination of these two components provides a compound which reduces nicotine cravings while simultaneously reducing appetite.

In addition to the two primary components, namely sulfur compounds used to block nicotine receptor cells, and the 5-HTP, which is used to increase serotonin levels of a variety of optional additional components can be used to enhance performance of the product. For example, weight suppression can be further enhanced through the use of Inositol, Vitamin B12 (as Methylcobalamin), L-methionine, and Choline Citrate. They can be used alone or in combination with one another to increase the body's ability to metabolize fat, and thereby helping the body to avoid weight gain. B vitamins, such as vitamin B-6 (as Pyridoxine HCl), Pantothenic Acid (as D-Calcium Pantothenate), and Folic Acid are precursors to, or co-factors for neurotransmitters, which will decrease cravings for certain carbohydrates. Alpha Lipoic Acid is helpful in stabilizing blood sugar levels. Magnesium Citrate may help to reduce anxiety.

Those skilled in the art will recognize that as is the case with any pharmaceutical or neutraceutical, appropriate dosages will vary based on several factors. A person's weight, age, physical condition, etc., will all influence what the proper dosage for a particular individual should be. As a result, while the foregoing dosages are envisioned as an appropriate starting point, changes can be made to suit particular individuals.

While the invention has been described with respect to a preferred embodiment thereof, it will be understood by those skilled in the art that various changes in detail may be made therein without departing from the spirit, scope, and teaching of the invention. For example, the compound used to suppress appetite can vary so long as it is suitable for its purpose, the amount of the appetite suppressant can vary based on an individual's physical requirements. The type of administration can vary from pill/capsule form to liquid form, injection, transdermal patch, creams, gels, gums, lozenges, etc. In addition, the dose and frequency of administration can vary based on the number of daily meals, severity of withdrawal systems, the weight and condition of the individual, etc. Accordingly, the invention herein disclosed is to be limited only as specified in the following claims.

What is claimed is:

1. A method of simultaneously treating tobacco/nicotine addiction, and weight control side effects caused by nicotine withdrawal by reducing the craving for nicotine while simultaneously suppressing increases in appetite caused by nicotine withdrawal, including the steps of:
combining a first compound having a nicotine craving suppressant with a second compound having at least one appetite suppressant into a treatment dosage; and
administering the treatment dosage to an individual at predetermined times, such that food cravings are reduced in the individual's body during the process of withdrawal from nicotine addiction.

2. A method, as in claim 1, including the additional step of:
reducing food cravings by increasing serotonin levels in the individual's body.

3. A method, as in claim 2, including the additional step of:
providing a serotonin precursor to assist individuals natural production of serotonin and individuals body.

4. A method, as in claim 3, including the additional step of:
using amino acids as the serotonin precursor.

5. A method, as in claim 4, including the additional step of:
using amino acids which include tryptophan derivatives.

6. A method, as in claim 5, including the additional step of:
using 5-HTP as at least one of the tryptophan derivatives.

7. A method, as in claim 6, including the additional step of:
administering the treatment dosage via pill/capsules, liquids, injections, transdermal patchs, creams, gels, gums, or lozenges.

8. A method, as in claim 2, including the additional steps:
using serotonin precursors derived from tryptophan to increase the serotonin levels; and
using at least one bivalent negative sulfur compound to reduce craving for nicotine.

9. A method, as in claim 8, including the additional step of:
using at least one bivalent negative sulfur compound from the group of hydropersulfides, alkyl sulfides, colloidal sulfur, organic thio compounds or their pharmaceutically acceptable salts.

10. A method, as in claim 9, wherein:
the bivalent negative sulfur compound or compounds are administered with a therapeutic dosage level of approximately 120 mg or more.

11. A method, as in claim 9, including the additional step of:
using thioglycerols, thioglycols or their pharmaceutically acceptable salts as the organic thio compounds.

12. A method, as in claim 11, including the additional steps of:
including approximately 200 mg of 5-HTP in the treatment dosage as a serotonin precursor; and
Including approximately 120 mg of thioglycerols, thioglycols or their pharmaceutically acceptable salts as a nicotine craving suppressant.

13. A method, as in claim 12, including the additional steps of:
including at least one or more of the following group: approximately 50 mg Choline Citrate, approximately 50 mg Pantothenic Acid (as D-Calcium Pantothenate), approximately 400 mg Inositol, approximately 50 mg L-methionine, approximately 100 mg Alpha Lipoic Acid, approximately 100 mg Vitamin B-6 (as Pyridoxine HCl), approximately 100 mg Vitamin C, approximately 400 mcg Folic Acid, approximately 100 mcg Vitamin B12 (as Methylcobalamin), approximately 50 mg Magnesium Citrate, approximately 200-400 mg L-Tyrosine, approximately 200 mg N-acetyl tyrosine, and/or approximately 300 mg L or DL-phenylalanine.

14. A method, as in claim 8, including the additional step of:
using 5-HTP as at least one of the tryptophan derivatives.

15. A compound for simultaneous treatment of tobacco/nicotine addiction, and suppression of unwanted weight gain caused by nicotine withdrawal side effects, comprising:
a nicotine craving suppressant; and
at least one appetite suppressant having at least one serotonin precursor to increase serotonin levels in an individual's body;
wherein the nicotine cravings are reduced by the nicotine craving suppressant, and food cravings are reduced by increased serotonin levels in the individual's body.

16. A compound, as in claim 15, wherein:
the appetite suppressant is a tryptophan derivative.

17. A compound, as in claim 16, wherein:
the tryptophan derivative is 5-HTP.

18. A compound, as in claim 17, wherein:
the nicotine suppressant is a bivalent negative sulfur compound from the group of hydropersulfides, alkyl sulfides, colloidal sulfur, organic thio compounds or their pharmaceutically acceptable salts.

19. A compound, as in claim 18, wherein:
the appetite suppressant includes approximately 200 mg of 5-HTP; and
the nicotine suppressant includes approximately 120 mg of thioglycerols, thioglycols or their pharmaceutically acceptable salts.

20. a compound, as in claim 19, further comprising:
at least one or more of the following group: approximately 50 mg Choline Citrate, approximately 50 mg Pantothenic Acid (as D-Calcium Pantothenate), approximately 400 mg Inositol, approximately 50 mg L-methionine, approximately 100 mg Alpha Lipoic Acid, approximately 100 mg Vitamin B-6 (as Pyridoxine HCl), approximately 100 mg Vitamin C, approximately 400 mcg Folic Acid, approximately 100 mcg Vitamin Bi 2 (as Methylcobalamin), approximately 50 mg Magnesium Citrate, approximately 200-400 mg L-Tyrosine, approximately 200 mg N-acetyl tyrosine, and/or approximately 300 mg L or DL-phenylalanine.
